(19) United States
(12) Patent Application Publication
(10) Pub. No.: US 2010/0268225 A1
(43) Pub. Date: Oct. 21, 2010

(54) Methods for Image Analysis and Visualization of Medical Image Data Suitable for Use in Assessing Tissue Ablation and Systems and Methods for Controlling Tissue Ablation Using Same

(75) Inventors: Jonathan A. Coe, Denver, CO (US); Casey M. Ladtkow, Westminster, CO (US)

Correspondence Address: TYCO Healthcare Group LP;Attn: IP Legal, 5920 Longbow Drive, Mail Stop A36, Boulder, CO, 80301-3299, US

(73) Assignee: TYCO Healthcare Group LP

(21) Appl. No.: 12/761,267

(22) Filed: Apr. 15, 2010

Related U.S. Application Data
(60) Provisional application No. 61/169,556, filed on Apr. 15, 2009.

(51) Int. Cl. A61B 18/16; G06K 9/62

(52) U.S. Cl. 606/42; 382/131

(57) ABSTRACT

A method of image analysis includes the initial step of receiving a data set including image data. The image data represents a sequence of 2-D slice images. The method includes the steps of segmenting an object of interest from surrounding image data of each slice image based on a topographic growth rule and a pixel intensity value threshold with respect to a starting pixel, and rendering a volume of the object of interest using (x,y) coordinates corresponding to boundaries of the segmented object of interest.

### CROSS-REFERENCE TO RELATED APPLICATION

This application claims priority to, and the benefit of, U.S. Provisional Application Ser. No. 61/169,556 filed on Apr. 15, 2009, the disclosure of which is herein incorporated by reference in its entirety.

### BACKGROUND

1. Technical Field

The present disclosure relates to data analysis and visualization techniques, and, more particularly, to methods for image analysis and visualization of medical image data that are suitable for use in assessing biological tissue ablation, and systems and methods for controlling tissue ablation using the same.

2. Discussion of Related Art

Treatment of certain diseases requires the destruction of malignant tissue growths, e.g., tumors. Electromagnetic radiation can be used to heat and destroy tumor cells. Treatment may involve inserting ablation probes into tissues where cancerous tumors have been identified. Once the probes are positioned, electromagnetic energy is passed through the probes into surrounding tissue.

In the treatment of diseases such as cancer, certain types of tumor cells have been found to denature at elevated temperatures that are slightly lower than temperatures normally injurious to healthy cells. Known treatment methods, such as hyperthermia therapy, heat diseased cells to temperatures above 41° C. while maintaining adjacent healthy cells below the temperature at which irreversible cell destruction occurs. These methods involve applying electromagnetic radiation to heat, ablate and/or coagulate tissue. Microwave energy is sometimes utilized to perform these methods. Other procedures utilizing electromagnetic radiation to heat tissue also include coagulation, cutting and/or ablation of tissue. Many procedures and types of devices utilizing electromagnetic radiation to heat tissue have been developed.

Medical imaging has become a significant component in the clinical setting and in basic physiology and biology research, e.g., due to enhanced spatial resolution, accuracy and contrast mechanisms that have been made widely available. Medical imaging now incorporates a wide variety of modalities that noninvasively capture the structure and function of the human body. Such images are acquired and used in many different ways including medical images for diagnosis, staging and therapeutic management of malignant disease.

Because of their anatomic detail, computed tomography (CT) and magnetic resonance imaging (MRI) are suitable for, among other things, evaluating the proximity of tumors to local structures. CT and MRI scans produce two-dimensional (2-D) axial images, or slices, of the body that may be viewed sequentially by radiologists who visualize or extrapolate from these views actual three-dimensional (3-D) anatomy.

Medical image processing, analysis and visualization play an increasingly significant role in many fields of biomedical research and clinical practice. While images of modalities such as MRI or CT may be displayed as 2-D slices, three-dimensional visualization of images and quantitative analysis requires explicitly defined object boundaries. For example, to generate a 3-D rendering of a tumor from a MRI image, the tumor needs to be first identified within the image and then the tumor's boundary marked and used for 3-D rendering. Measurements and quantitative analysis for parameters such as area, perimeter, volume and length may be obtained when object boundaries are defined.

A boundary in an image is a contour that represents the change from one object or surface to another. Image segmentation involves finding salient regions and their boundaries. A number of image segmentation methods have been developed using fully automatic or semi-automatic approaches for medical imaging and other applications. Medical image segmentation refers to the delineation of anatomical structures and other regions of interest in medical images for assisting doctors in evaluating medical imagery or in recognizing abnormal findings in a medical image. Structures of interest may include organs or parts thereof, such as cardiac ventricles or kidneys, abnormalities such as tumors and cysts, as well as other structures such as bones and vessels. Despite the existence of numerous image segmentation techniques, segmentation of medical images is still a challenge due to the variety and complexity of medical images.

Medical image analysis and visualization play an increasingly significant role in disease diagnosis and monitoring as well as, among other things, surgical planning and monitoring of therapeutic procedures. Three-dimensional image visualization techniques may be used to provide the clinician with a more complete view of the anatomy, reducing the variability of conventional 2-D visualization techniques. Three-dimensional visualization of medical images of modalities such as CT or MRI may facilitate planning and effective execution of therapeutic hyperthermic treatments.

### SUMMARY

The present disclosure relates to a method of image analysis including the initial step of receiving a data set including image data. The image data represents a sequence of 2-D slice images. The method includes the steps of segmenting an object of interest from surrounding image data of each slice image based on a topographic growth rule and a pixel intensity value threshold with respect to a starting pixel, and rendering a volume of the object of interest using (x,y) coordinates corresponding to boundaries of the segmented object of interest.

The present disclosure relates to a method of image analysis including the initial step of receiving a data set including image data. The image data represents a sequence of 2-D slice images. The method includes the steps of selectively defining a region of interest within each slice image of the sequence of 2-D slice images, characterizing pixels contained within the region of interest of each slice image based on statistical properties derived from pixel values within the region of interest of each slice image, and segmenting an object of interest from surrounding image data of each slice image based on a topographic growth rule and a pixel intensity value threshold with respect to a starting pixel, wherein the pixel intensity value threshold is based on the derived statistical properties. The method also includes the steps of determining (x,y) coordinates corresponding to boundaries of the segmented object of interest of each slice image, arranging the boundaries of the segmented object of interest of each slice image along a third dimension using the (x,y) coordinates corresponding to the plurality of 2-D slice images, and fitting a 3-D surface to the arranged boundaries to render an approximate volume of the object of interest.

The present disclosure also relates to a method of directing energy to tissue including the initial step of positioning an energy applicator for delivery of energy to tissue. The energy applicator is operably associated with an electrosurgical power generating source. The method includes the steps of determining at least one operating parameter associated with the electrosurgical power generating source based on at least one parameter of a rendered ablation volume, and transmitting energy from the electrosurgical power generating source through the energy applicator to tissue.

### BRIEF DESCRIPTION OF THE DRAWINGS

Objects and features of the presently disclosed methods for image analysis and visualization of medical image data and the presently disclosed systems and methods for controlling tissue ablation using the same will become apparent to those of ordinary skill in the art when descriptions of various embodiments thereof are read with reference to the accompanying drawings, of which:

FIG. 1 is a schematic illustration of an ablation system including an energy applicator positioned for the delivery of energy to a targeted tissue area according to an embodiment of the present disclosure;

FIG. 2 is a diagrammatic representation of a two-dimensional (2-D) image slice showing patient tissue surrounding an object of interest according to an embodiment of the present disclosure;

FIG. 3 is a diagrammatic representation of the 2-D image slice of FIG. 2 showing a user-defined region of interest shown by a dashed circle within the object of interest according to an embodiment of the present disclosure;

FIG. 4 is a diagrammatic representation of a thresholded image of the 2-D image slice of FIG. 2 according to an embodiment of the present disclosure;

FIG. 5 is a diagrammatic representation of a resulting image of topographical rule based processing showing the segmented object of interest of FIG. 4 according to an embodiment of the present disclosure;

FIGS. 6A and 6B are diagrammatic representations of morphological dilation and erosion operations on the object of interest of FIG. 5 according to an embodiment of the present disclosure;

FIG. 7 is a schematic view of a volume rendered ablation according to an embodiment of the present disclosure;

FIG. 8 is a schematic view of a volume rendered ablation according to an embodiment of the present disclosure;

FIG. 9 is a flowchart illustrating a method of image analysis according to an embodiment of the present disclosure; and

FIG. 10 is a flowchart illustrating a method of directing energy to tissue according to an embodiment of the present disclosure.

### DETAILED DESCRIPTION

Hereinafter, embodiments of presently disclosed methods for image analysis and visualization of medical image data and the presently disclosed systems and methods for controlling tissue ablation using the same are described with reference to the accompanying drawings. Like reference numerals may refer to similar or identical elements throughout the description of the figures. As shown in the drawings and as used in this description, and as is traditional when referring to relative positioning on an object, the term “proximal” refers to that portion of the object that is closer to the user and the term “distal” refers to that portion of the object that is farther from the user.

This description may use the phrases “in an embodiment,” “in embodiments,” “in some embodiments,” or “in other embodiments,” which may each refer to one or more of the same or different embodiments in accordance with the present disclosure. For the purposes of this description, a phrase in the form “A/B” means A or B. For the purposes of the description, a phrase in the form “A and/or B” means “(A), (B), or (A and B)”. For the purposes of this description, a phrase in the form “at least one of A, B, or C” means “(A), (B), (C), (A and B), (A and C), (B and C), or (A, B and C)”.

Electromagnetic energy is generally classified by increasing energy or decreasing wavelength into radio waves, microwaves, infrared, visible light, ultraviolet, X-rays and gamma-rays. As it is used in this description, “microwave” generally refers to electromagnetic waves in the frequency range of 300 megahertz (MHz) (3×10⁸ cycles/second) to 300 gigahertz (GHz) (3×10¹¹ cycles/second). As it is used in this description, “ablation procedure” generally refers to any ablation procedure, such as microwave ablation, radio frequency (RF) ablation or microwave ablation assisted resection. As it is used in this description, “energy applicator” generally refers to any device that can be used to transfer energy from a power generating source, such as a microwave or RF electrosurgical generator, to tissue. As it is used in this description, “transmission line” generally refers to any transmission medium that can be used for the propagation of signals from one point to another.

As it is used in this description, “length” may refer to electrical length or physical length. In general, electrical length is an expression of the length of a transmission medium in terms of the wavelength of a signal propagating within the medium. Electrical length is normally expressed in terms of wavelength, radians or degrees. For example, electrical length may be expressed as a multiple or sub-multiple of the wavelength of an electromagnetic wave or electrical signal propagating within a transmission medium. The wavelength may be expressed in radians or in artificial units of angular measure, such as degrees. The electric length of a transmission medium may be expressed as its physical length multiplied by the ratio of (a) the propagation time of an electrical or electromagnetic signal through the medium to (b) the propagation time of an electromagnetic wave in free space over a distance equal to the physical length of the medium. The electrical length is in general different from the physical length. By the addition of an appropriate reactive element (capacitive or inductive), the electrical length may be made significantly shorter or longer than the physical length.

Various embodiments of the present disclosure provide systems and methods of directing energy to tissue. Embodiments may be implemented using electromagnetic radiation at microwave frequencies or at other frequencies. An electromagnetic energy delivery device including an energy applicator array, according to various embodiments, is designed and configured to operate between about 300 MHz and about 10 GHz.

Various embodiments of the presently disclosed electrosurgical system including an energy applicator, or energy applicator array, are suitable for microwave ablation and for use to pre-coagulate tissue for microwave ablation assisted surgical resection. In addition, although the following description describes the use of a dipole microwave antenna, the teachings of the present disclosure may also apply to a monopole, helical, or other suitable type of microwave antenna.

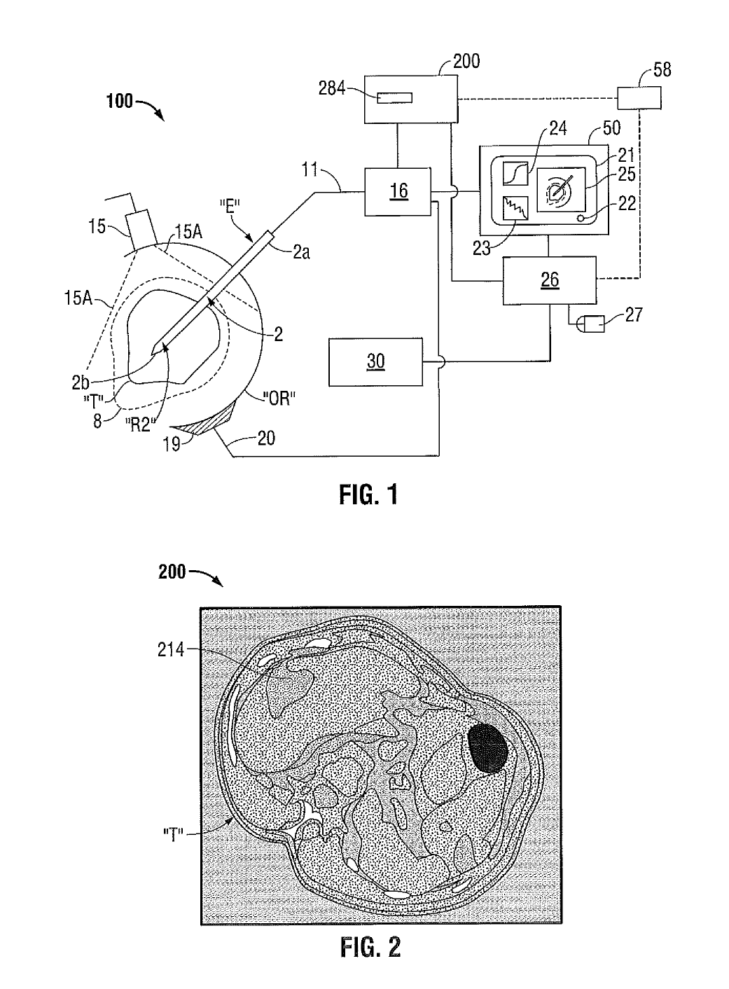

An electrosurgical system 100 according to an embodiment of the present disclosure is shown in FIG. 1 and includes an electromagnetic energy delivery device or energy applicator array “E”. Energy applicator array “E” includes an energy applicator or probe 2. As one of ordinary skill in the art will readily recognize, other energy applicator array “E” embodiments may include a plurality of energy applicators.

Probe 2 generally includes a radiating section “R1” operably connected by a feedline (or shaft) 2a to an electrosurgical power generating source 16, e.g., a microwave or RF electrosurgical generator. Power generating source 16 may be configured to provide various frequencies of electromagnetic energy. A transmission line 11 may be provided to electrically couple the feedline 2a to the electrosurgical power generating source 16.

Feedline 2a may be formed from a suitable flexible, semi-rigid or rigid microwave conductive cable, and may connect directly to an electrosurgical power generating source 16. Feedline 2a may have a variable length from a proximal end of the radiating section “R2” to a distal end of the transmission line 11 ranging from a length of about one inch to about twelve inches. Transmission line 11 may additionally, or alternatively, provide a conduit (not shown) configured to provide coolant fluid from a coolant source (not shown) to the energy applicator array “E”.

Located at the distal end of the probe 2 is a tip portion 2b, which may be configured to be inserted into an organ “OR” of a human body or any other body tissue. Tip portion 2b may terminate in a sharp tip to allow for insertion into tissue with minimal resistance. Tip portion 2b may include other shapes, such as, for example, a tip that is rounded, flat, square, hexagonal, or cylindroconical.

Electrosurgical system 100 includes a user interface 50. User interface 50 may include a display 21, such as without limitation a flat panel graphic LCD (liquid crystal display), adapted to visually display one or more user interface elements 23, 24, 25. In an embodiment, the display 21 includes touchscreen capability (not shown), e.g., the ability to receive input from an object in physical contact with the display 21, such as without limitation a stylus or a user's fingertip. A user interface element 23, 24, 25 may have a corresponding active region, such that, by touching the screen within the active region associated with the user interface element, an input associated with the user interface element 23, 24, 25 is received by the user interface 50.

User interface 50 may additionally, or alternatively, include one or more controls 22 that may include without limitation a switch (e.g., pushbutton switch, toggle switch, slide switch) and/or a continuous actuator (e.g., rotary or linear potentiometer, rotary or linear encoder). In an embodiment, a control 22 has a dedicated function, e.g., display contrast, power on/off, and the like. Control 22 may also have a function that may vary in accordance with an operational mode of the electrosurgical system 100. A user interface element (e.g., 23 shown in FIG. 1) may be provided to indicate the function of the control 22. Control 22 may also include an indicator, such as an illuminated indicator, e.g., a single- or variably-colored LED (light emitting diode) indicator.

As shown in FIG. 1, the electrosurgical system 100 may include a reference electrode 19 (also referred to herein as a “return” electrode). Return electrode 19 may be electrically coupled via a transmission line 20 to the power generating source 16. During a procedure, the return electrode 19 may be positioned in contact with the skin of the patient or a surface of the organ “OR”. When the surgeon activates the energy applicator array “E”, the return electrode 19 and the transmission line 20 may serve as a return current path for the current flowing from the power generating source 16 through the probe 2.

During microwave ablation using the electrosurgical system 100 the energy applicator array “E” is inserted into or placed adjacent to tissue and microwave energy is supplied thereto. Ultrasound or computed tomography (CT) guidance may be used to accurately guide the energy applicator array “E” into the area of tissue to be treated. Probe 2 may be placed percutaneously or surgically, e.g., using conventional surgical techniques by surgical staff. A clinician may pre-determine the length of time that microwave energy is to be applied. Application duration may depend on a variety of factors such as energy applicator design, number of energy applicators used simultaneously, tumor size and location, and whether the tumor was a secondary or primary cancer. The duration of microwave energy application using the energy applicator array “E” may depend on the progress of the heat distribution within the tissue area that is to be destroyed and/or the surrounding tissue.

FIG. 1 shows a targeted region including ablation targeted tissue represented in sectional view by the solid line “T”. It may be desirable to ablate the targeted region “T” by fully engulfing the targeted region “T” in a volume of lethal heat elevation. Targeted region “T” may be, for example, a tumor that has been detected by a medical imaging system 30.

Medical imaging system 30, according to various embodiments, includes a scanner (e.g., 15 shown in FIG. 1) of any suitable imaging modality, or other image acquisition device capable of generating input pixel data representative of an image. Medical imaging system 30 may additionally, or alternatively, include a medical imager operable to form a visible representation of the image based on the input pixel data. Medical imaging system 30 may include a storage device such as an internal memory unit, which may include an internal memory card and removable memory. In some embodiments, the medical imaging system 30 may be a multi-modal imaging system capable of scanning using different modalities. Examples of imaging modalities that may be suitably and selectively used include X-ray systems, ultrasound (UT) systems, magnetic resonance imaging (MRI) systems, computed tomography (CT) systems, single photon emission computed tomography (SPECT), and positron emission tomography (PET) systems. Medical imaging system 30 may include any device capable of generating digital data representing an anatomical region of interest. In some embodiments, the medical imaging system 30 includes a MRI scanner and/or a CT scanner capable of generating two-dimensional (2-D) image slices (e.g., 200 shown in FIG. 12).

Image data representative of one or more images may be communicated between the medical imaging system 30 and a processor unit 26. Medical imaging system 30 and the processor unit 26 may utilize wired communication and/or wireless communication. Processor unit 26 may include any type of computing device, computational circuit, or any type of processor or processing circuit capable of executing a series of instructions that are stored in a memory (not shown) associated with the processor unit 26. Processor unit 26 may be adapted to run an operating system platform and application programs. A scanner (e.g., 15 shown in FIG. 1) of any suitable imaging modality may additionally, or alternatively, be disposed in contact with the organ “OR” to provide image data. As an illustrative example, the two dashed lines 15A in FIG. 1 bound a region for examination by the scanner 15, e.g., a CT scanner.

In FIG. 1, the dashed line 8 surrounding the targeted region “T” represents the ablation isotherm in a sectional view through the organ “OR”. Such an ablation isotherm may be that of the surface achieving possible temperatures of approximately 50° C. or greater. The shape and size of the ablation volume, as illustrated by the dashed line 8, may be influenced by a variety of factors including the configuration of the energy applicator array “E”, the geometry of the radiating section “R2”, cooling of the probe 2, ablation time and wattage, and tissue characteristics. Processor unit 26 may be connected to one or more display devices (e.g., 21 shown in FIG. 1) used by the clinician to visualize the targeted region “T” and/or the ablation volume 8 during a procedure, e.g., an ablation procedure.

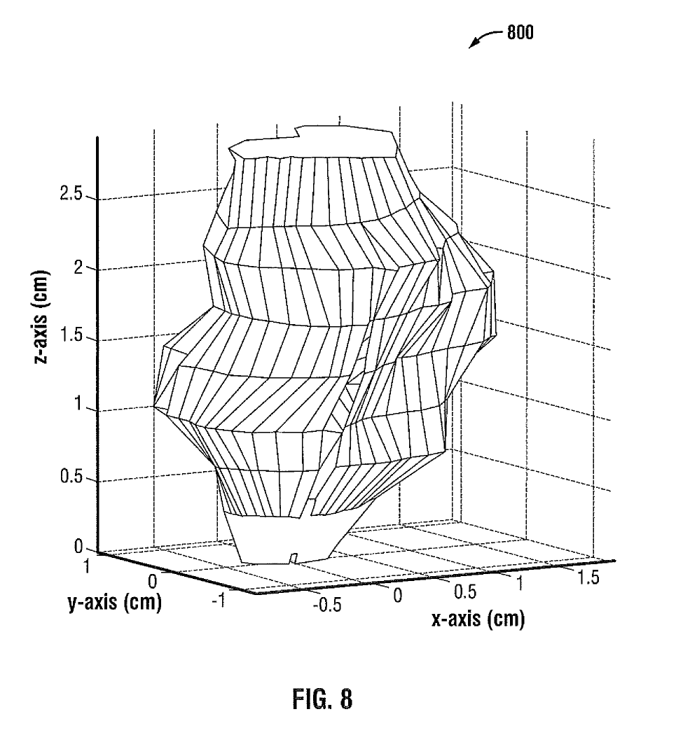

Electrosurgical system 100 may include a library 200. As it is used in this description, “library” generally refers to any repository, databank, database, cache, storage unit and the like. Library 200 may include a database 284 that is configured to store and retrieve energy applicator data, e.g., parameters associated with one or energy applicators and/or one or more energy applicator arrays. Parameters stored in the database 284 in connection with an energy applicator array may include, but are not limited to, energy applicator array identifier, energy applicator array dimensions, a frequency, an ablation length, an ablation diameter, a temporal coefficient, a shape metric, and/or a frequency metric. Volume rendered ablations (e.g., 700 and 800 shown in FIGS. 7 and 8, respectively) may be stored in the database 284. In an embodiment, ablation pattern topology may be included in the database 284, e.g., a wireframe model of an energy applicator array (e.g., 25 shown in FIG. 1) and/or a representation of a radiation pattern associated therewith.

Library 200 according to embodiments of the present disclosure may be communicatively associated with a picture archiving and communication systems (PACS) database (shown generally as 58 in FIG. 1) containing DICOM (acronym for Digital Imaging and Communications in Medicine) formatted medical images. PACS database 58 may be configured to store and retrieve image data, e.g., representing a sequence of 2-D slice images, from a variety of imaging modalities. As shown in FIG. 1, the processor unit 26 may be communicatively associated with the PACS database 58. In accordance with one or more presently-disclosed methods, image data associated with a prior treatment of a target tissue volume is retrieved from the PACS database 58 and the ablation volume is rendered using a sequence of 2-D image slices of the image data.

Images and/or non-graphical data stored in the library 200, and/or retrievable from the PACS database 58, may be used to configure the electrosurgical system 100 and/or control operations thereof. For example, volume rendered ablations (e.g., 700 and 800 shown in FIGS. 7 and 8, respectively) associated with an energy applicator, according to embodiments of the present disclosure, may be used as a feedback tool to control an instrument's and/or clinician's motion, e.g., to allow clinicians to avoid ablating critical structures, such as large vessels, healthy organs or vital membrane barriers.

Images and/or non-graphical data stored in the library 200, and/or retrievable from the PACS database 58, such as volume rendered ablations (e.g., 700 and 800 shown in FIGS. 7 and 8, respectively) may be used to facilitate planning and effective execution of a procedure, e.g., an ablation procedure. Images and/or information displayed on the display 21 of the user interface 50, for example, may be used by the clinician to better visualize and understand how to achieve more optimized results during thermal treatment of tissue, such as, for example, ablation of tissue, tumors and cancer cells.

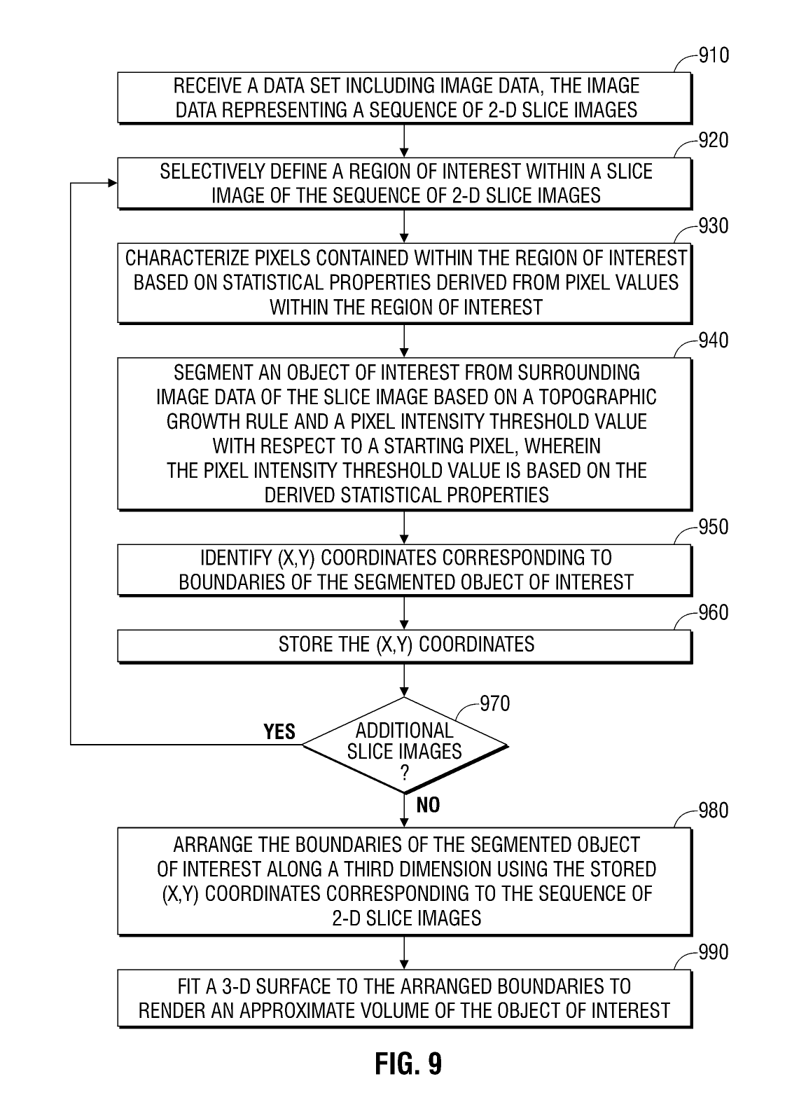

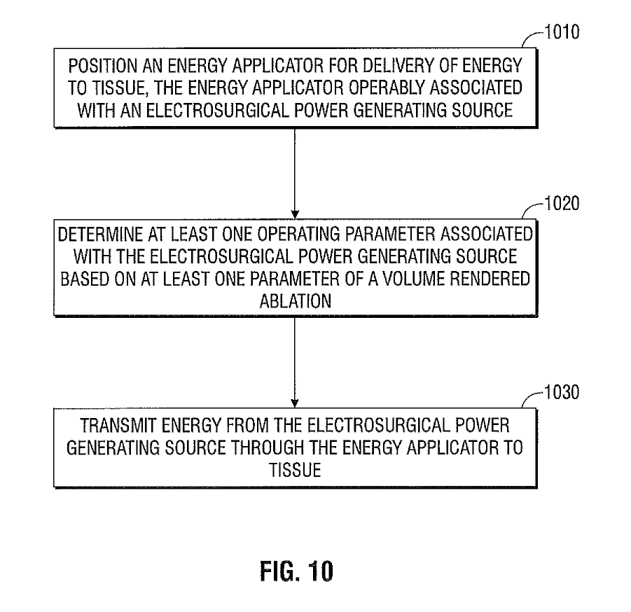

Hereinafter, a method of image analysis is described with reference to FIG. 9, and a method of directing energy to tissue is described with reference to FIG. 10. It is to be understood that the steps of the methods provided herein may be performed in combination and in a different order than presented herein without departing from the scope of the disclosure.

FIG. 9 is a flowchart illustrating a method of image analysis according to an embodiment of the present disclosure. In step 910, a data set including image data is received. The image data represents a sequence of 2-D slice images.

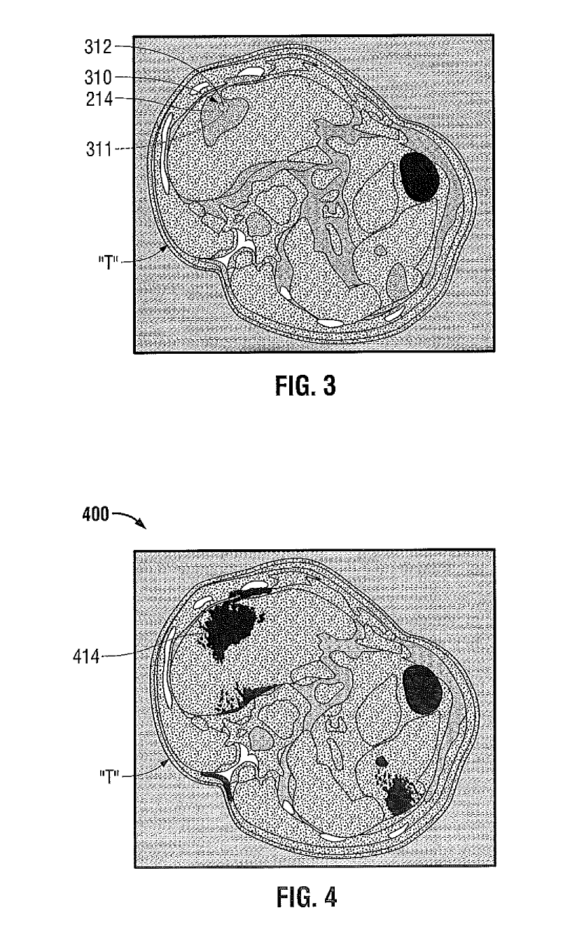

In step 920, a region of interest is selectively defined within a slice image (e.g., 200 shown in FIG. 2) of the sequence of 2-D slice images. In FIG. 3, the dashed circle 311 is a user-defined region of interest. Referring to FIG. 1, the clinician may use a pointing device 27 coupled to the processor unit 26 and/or the touchscreen capability of the display 21 of the electrosurgical system 100 to create a circle, or other shape, around a selected region of interest (e.g., 310 shown in FIG. 3) within an object of interest (e.g., 214 shown in FIGS. 2 and 3).

In step 930, pixels contained within the region of interest are characterized based on statistical properties derived from pixel values within the region of interest. In embodiments, the mean pixel values and their standard deviations are measured within the region of interest (e.g., 310 shown in FIG. 3). In embodiments, the pixel intensity threshold is any pixel in the entire image that is within a mean +/− a multiple of the standard deviation that was characterized to be the region of interest. In one embodiment, the multiple of the standard deviation is 1.8.

In step 940, an object of interest (e.g., 214 shown in FIGS. 2 and 3) is segmented from the surrounding image data based on a topographic growth rule and a pixel intensity value threshold with respect to a starting pixel. The pixel intensity value threshold is based on the statistical properties derived in step 930. The starting pixel (e.g., 312 shown in FIG. 3) may be automatically selected, e.g., using knowledge of an anatomical structure or a region of interest. The starting pixel may be user-defined, and may be a pixel located at or near the center of a user-defined shape, e.g., the dashed circle 311 shown in FIG. 3. A method of region growing, according to an embodiment of the present disclosure, is used to basically only select pixels that are within a certain range of the starting pixel value. A topographic growth rule is applied requiring that the pixels are adjacent and that the pixels are within a certain grey value of each other. Starting at the starting pixel, the presently disclosed method of region growing sequentially looks for the adjacent pixels and includes those pixels that are within a predetermined grey value of each other.

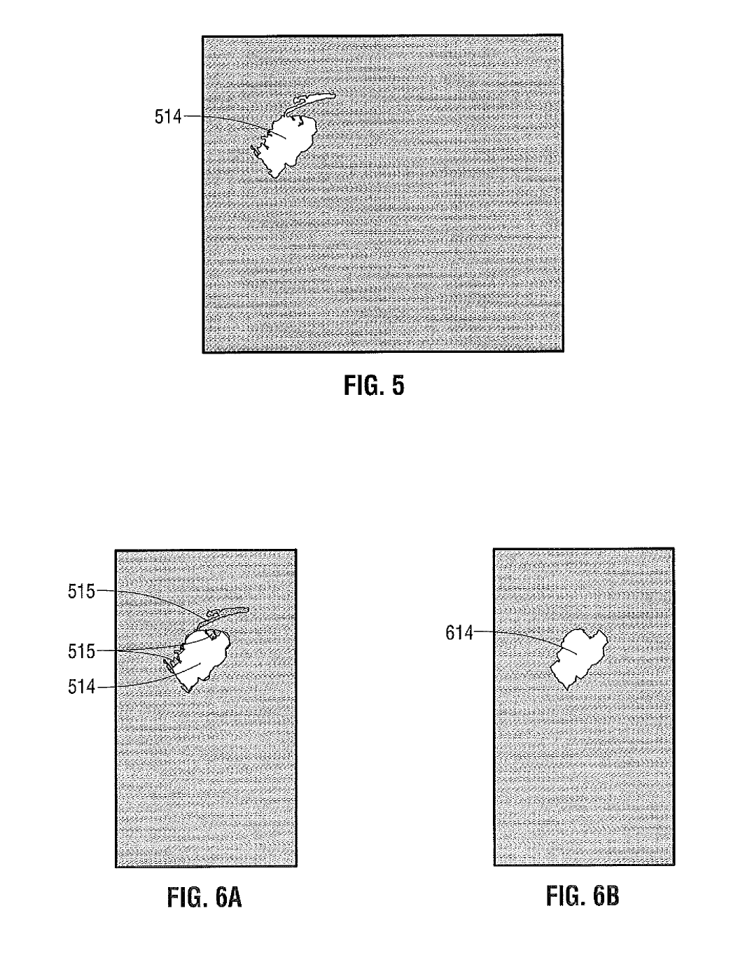

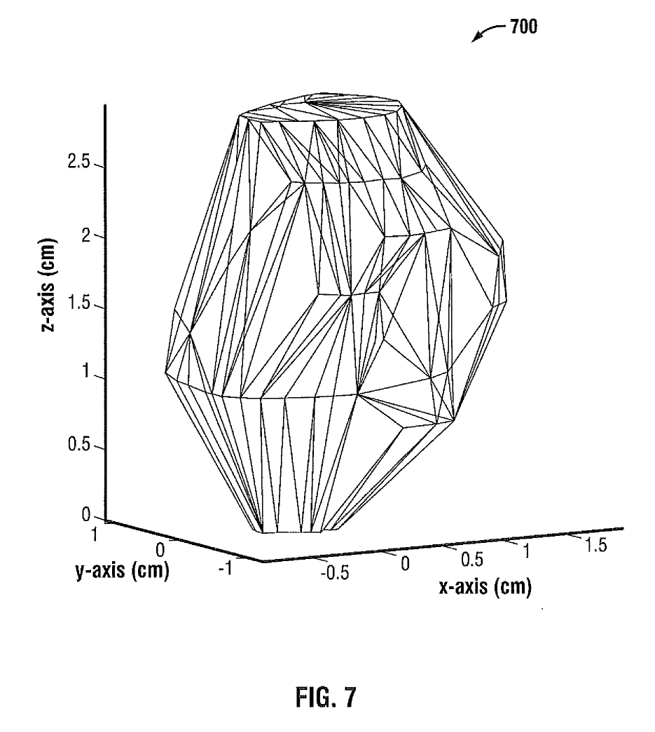

FIG. 4 shows a thresholded 2-D image slice that includes a thresholded object of interest 414 (e.g., an ablation) according to an embodiment of the present disclosure. FIG. 5 shows the segmented object of interest 514 resulting from topographical rule-based processing of the region of interest of FIG. 4 according to an embodiment of the present disclosure. FIGS. 6A and 6B show an object of interest 614 resulting from morphological dilation and erosion operations on the object of interest 514 of FIG. 5 that discarded stringers 515 according to an embodiment of the present disclosure.

In step 950, (x,y) coordinates corresponding to the boundaries of the segmented object of interest are determined. The (x,y) coordinates are stored in a memory, in step 960.

In step 970, it is determined whether additional slice images remain to be processed. If it is determined that there are additional slice images, then repeat step 920 through step 960, as described above.

If it is determined that there are no additional slice images, then, in step 980, the boundaries of the segmented object of interest are arranged along a third dimension using the stored (x,y) coordinates corresponding to the plurality of 2-D slice images. The spacing of this arrangement along the third dimension (e.g., z-axis shown in FIGS. 7 and 8) is preferably equal to the anatomic spacing of the medical images.

In step 990, a 3-D surface is fitted to the arranged boundaries to render an approximate volume of the object of interest. In some embodiments, quantitative analysis may be performed for determining the size, density and other parameters of the volume rendered object of interest. Data associated with the object of interest (e.g., 700 and 800 shown in FIGS. 7 and 8, respectively) may be stored in a database (e.g., 284 shown in FIG. 1), and may be used for controlling an ablation procedure.

FIG. 10 is a flowchart illustrating a method of directing energy to tissue according to an embodiment of the present disclosure. In step 1010, an energy applicator (e.g., “E” shown in FIG. 1) is positioned for delivery of energy to tissue (e.g., “T” shown in FIG. 1), wherein the energy applicator is operably associated with an electrosurgical power generating source (e.g., 16 shown in FIG. 1).

In step 1020, at least one operating parameter associated with the electrosurgical power generating source is determined based at least one parameter of a rendered ablation volume. Examples of operating parameters associated with the electrosurgical power generating source include without limitation temperature, impedance, power, current, voltage, mode of operation, and duration of application of electromagnetic energy. Examples of parameters of a volume rendered ablation (e.g., 700 and 800 shown in FIGS. 7 and 8, respectively) include without limitation volume, length, diameter, minimum diameter, maximum diameter and centroid. Volume rendered ablations may be stored in a database (e.g., 284 shown in FIG. 1) prior to and/or during a procedure. In embodiments, the volume rendered ablation is generated from image data representing a sequence of 2-D slice images, e.g., in accordance with the presently disclosed image analysis method illustrated in FIG. 9.

In step 1030, energy from the electrosurgical power generating source is transmitted through the energy applicator to tissue. The duration of energy application using the energy applicator may depend on the progress of the heat distribution within the tissue area that is to be destroyed and/or the surrounding tissue. In some embodiments, the duration of energy application using the energy applicator may depend on, among other things, the volume of a volume rendered ablation, e.g., stored in a database.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. A method of image analysis, comprising the steps of:
receiving a data set including image data, the image data representing a sequence of 2-D slice images;
segmenting an object of interest from surrounding image data of each slice image based on a topographic growth rule and a pixel intensity value threshold with respect to a starting pixel; and
rendering a volume of the object of interest using (x,y) coordinates corresponding to boundaries of the segmented object of interest.

2. The method of image analysis in accordance with claim 1, wherein rendering the volume of the object of interest using (x,y) coordinates corresponding to boundaries of the segmented object of interest includes the steps of:
determining (x,y) coordinates corresponding to boundaries of the segmented object of interest of each slice image;
arranging the boundaries of the segmented object of interest of each slice image along a third dimension using the (x,y) coordinates corresponding to the plurality of 2-D slice images; and
fitting a 3-D surface to the arranged boundaries to render a volume of the object of interest.

3. The method of image analysis in accordance with claim 1, wherein segmenting an object of interest from surrounding image data of each slice image based on a topographic growth rule and a pixel intensity value threshold with respect to a starting pixel includes the steps of:
selectively defining a region of interest within each slice image of the sequence of 2-D slice images; and
characterizing pixels contained within the region of interest of each slice image based on statistical properties derived from pixel values within the region of interest of each slice image.

4. The method of image analysis in accordance with claim 3, wherein the statistical properties include mean pixel values and their standard deviations.

5. The method of image analysis in accordance with claim 1, wherein the image data representing the sequence of 2-D slice images is in DICOM format.

6. The method of image analysis in accordance with claim 1, further comprising the step of:
displaying the rendered volume of the object of interest on a display device to facilitate planning of a procedure.

7. A method of image analysis, comprising the steps of:
receiving a data set including image data, the image data representing a sequence of 2-D slice images;
selectively defining a region of interest within each slice image of the sequence of 2-D slice images;
characterizing pixels contained within the region of interest of each slice image based on statistical properties derived from pixel values within the region of interest of each slice image;
segmenting an object of interest from surrounding image data of each slice image based on a topographic growth rule and a pixel intensity value threshold with respect to a starting pixel;
determining (x,y) coordinates corresponding to boundaries of the segmented object of interest of each slice image;
arranging the boundaries of the segmented object of interest of each slice image along a third dimension using the (x,y) coordinates corresponding to the plurality of 2-D slice images; and
fitting a 3-D surface to the arranged boundaries to render a volume of the object of interest.

8. The method of image analysis in accordance with claim 7, wherein the statistical properties include mean pixel values and their standard deviations.

9. The method of image analysis in accordance with claim 7, wherein the pixel intensity value threshold is based on the statistical properties.

10. The method of image analysis in accordance with claim 7, wherein the step of receiving a data set including image data includes retrieving image data from a picture archiving and communication system (PACS).

11. The method of image analysis in accordance with claim 7, further comprising the step of:
displaying the rendered volume of the object of interest on a display device to facilitate planning of a procedure.

12. The method of image analysis in accordance with claim 7, wherein selectively defining the region of interest includes the steps of:
displaying image data on a display device; and
providing a pointing device to enable user selection of the region of interest.

13. A method of directing energy to tissue, comprising the steps of:
positioning an energy applicator for delivery of energy to tissue, the energy applicator operably associated with an electrosurgical power generating source;
determining at least one operating parameter associated with the electrosurgical power generating source based on at least one parameter of a rendered ablation volume; and
transmitting energy from the electrosurgical power generating source through the energy applicator to tissue.

14. The method of directing energy to tissue in accordance with claim 13, wherein the at least one operating parameter associated with the electrosurgical power generating source is selected from the group consisting of temperature, impedance, power, current, voltage, mode of operation, and duration of application of electromagnetic energy.

15. The method of directing energy to tissue in accordance with claim 13, wherein the at least one parameter of a rendered ablation volume is selected from the group consisting of volume, length, diameter, minimum diameter, maximum diameter and centroid.

16. The method of directing energy to tissue in accordance with claim 13, wherein the rendered ablation volume is stored in a database.

17. The method of directing energy to tissue in accordance with claim 13, wherein the rendered ablation volume is generated from image data representing a sequence of 2-D slice images.
